(19) United States
(12) Patent Application Publication
(10) Pub. No.: US 2005/0209244 A1
(43) Pub. Date: Sep. 22, 2005

(54) N{5-[4-(4-methyl-piperazino-methyl)-benzoylamido]-2-methylphenyl}-4-(3-pyridyl)-2-pyrimidine-amine coated stents

(76) Inventors: Margaret Forney Prescott, Millburn, NJ (US); David Louis Feldman, Teaneck, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/505,398

(22) PCT Filed: Feb. 27, 2003
(86) PCT No.: PCT/EP03/02028

§ 371 Date: Apr. 29, 2005

Related U.S. Application Data
(60) Provisional application No. 60/360,254, filed on Feb. 28, 2002.

(51) Int. Cl. A61K 31/506; A61F 2/00

(52) U.S. Cl. 514/252.18; 424/423

(57) ABSTRACT

The invention relates to the local administration of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with one or more other active ingredients, and a device adapted for such local administration.

The present invention relates to drug delivery systems for the prevention and treatment of proliferative diseases, particularly vascular diseases. The invention furthermore relates to the use of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, for stabilizing vulnerable plaques in blood vessels of a subject in need of such a stabilization, for preventing or treating restenosis in diabetic patients, or for the prevention or reduction of vascular access dysfunction in association with the insertion or repair of an indwelling shunt, fistula or catheter in a subject in need thereof.

Many humans suffer from circulatory diseases caused by a progressive blockage of the blood vessels that perfuse the heart and other major organs. Severe blockage of blood vessels in such humans often leads to ischemic injury, hypertension, stroke or myocardial infarction. Atherosclerotic lesions which limit or obstruct coronary or periphery blood flow are the major cause of ischemic disease related morbidity and mortality including coronary heart disease and stroke. To stop the disease process and prevent the more advanced disease states in which the cardiac muscle or other organs are compromised, medical revascularization procedures such as percutaneous transluminal coronary angioplasty (PCTA), percutaneous transluminal angioplasty (PTA), atherectomy, bypass grafting or other types of vascular grafting procedures are used.

Re-narrowing (e.g. restenosis) of an artherosclerotic coronary artery after various revascularization procedures occurs in 10-80% of patients undergoing this treatment, depending on the procedure used and the aterial site. Besides opening an artery obstructed by atherosclerosis, revascularization also injures endothelial cells and smooth muscle cells within the vessel wall, thus initiating a thrombotic and inflammatory response. Cell derived growth factors such as platelet derived growth factor, Infiltrating macrophages, leukocytes or the smooth muscle cells themselves provoke proliferative and migratory responses in the smooth muscle cells. Simultaneous with local proliferation and migration, inflammatory cells also invade the site of vascular injury and may migrate to the deeper layers of the vessel wall. Proliferation/migration usually begins within one to two days post-injury and, depending on the revascularization procedure used, continues for days and weeks.

Both cells within the atherosclerotic lesion and those within the media migrate, proliferate and/or secrete significant amounts of extracellular matrix proteins. Proliferation, migration and extracellular matrix synthesis continue until the damaged endothelial layer is repaired at which time proliferation slows within the Intima. The newly formed tissue is called neointima, intimal thickening or restenotic lesion and usually results in narrowing of the vessel lumen. Further lumen narrowing may take place due to constructive remodeling, e.g. vascular remodeling, leading to further intimal thickening or hyperplasia.

Furthermore, there are also atherosclerotic lesions which do not limit or obstruct vessel blood flow but which form the so-called “vulnerable plaques”. Such atherosclerotic lesions or vulnerable plaques are prone to rupture or ulcerate, which results in thrombosis and thus produces unstable angina pectoris, myocardial infarction or sudden death. Inflamed atherosclerotic plaques can be detected by thermography.

Complications associated with vascular access devices is a major cause of morbidity in many disease states. For example, vascular access dysfunction in hemodialysis patients is generally caused by outflow stenoses in the venous circulation (Schwam S. J., et al., Kidney Int. 36: 707-711, 1989). Vascular access related morbidity accounts for about 23 percent of all hospital stays for advanced renal disease patients and contributes to as much as half of all hospitalization costs for such patients (Feldman H. I., J. Am. Soc. Nephrol. 7: 523-535, 1996). Additionally, vascular access dysfunction in chemotherapy patients is generally caused by outflow stenoses in the venous circulation and results in a decreased ability to administer medications to cancer patients. Often the outflow stenoses is so severe as to require intervention. Additionally, vascular access dysfunction in total parenteral nutrition (TPN) patients is generally caused by outflow stenoses in the venous circulation and results in reduced ability to care for these patients. Up to the present time, there has not been any effective drug for the prevention or reduction of vascular access dysfunction that accompany the insertion or repair of an indwelling shunt, fistula or catheter, such as a large bore catheter, into a vein in a mammal, particularly a human patient. Survival of patients with chronic renal failure depends on optimal regular performance of dialysis. If this is not possible (for example as a result of vascular access dysfunction or failure), it leads to rapid clinical deterioration and unless the situation is remedied, these patients will die. Hemodialysis requires access to the circulation. The ideal form of hemodialysis vascular access should allow repeated access to the circulation, provide high blood flow rates, and be associated with minimal complications. At present, the three forms of vascular access are native arteriovenous fistulas (AVF), synthetic grafts, and central venous catheters. Grafts are most commonly composed of polytetrafluoroethylene (PTFE, or Gore-Tex). Each type of access has its own advantages and disadvantages.

Vascular access dysfunction is the most important cause of morbidity and hospitalization in the hemodialysis population. Venous neointimal hyperplasia characterized by stenosis and subsequent thrombosis accounts for the overwhelming majority of pathology resulting in dialysis graft failure.

The most common form of vascular access procedure performed in chronic hemodialysis patients in the United States is the arteriovenous polytetrafluoroethylene (PTFE) graft, which accounts for approximately 70% of all hemodialysis access.

Dr. Burnett S. Kelly and Col., (Kidney International, Volume 62; Issue 6; Page 2272-December 2002) and others have previously shown that venous neointimal hyperplasia (VNH) in the setting of arteriovenous hemodialysis grafts is characterized by proliferation of smooth muscle cells, and the abundance of neointimal and adventitial microvessels and extracellular matrix components. However, despite a reasonable knowledge of the pathology of VNH, there are still no effective interventions for either the prevention or treatment of hemodialysis vascular access dysfunction. This is particularly unfortunate, as VNH in the setting of hemodialysis grafts appears to be a far more aggressive lesion as compared to the more common arterial neointimal hyperplasia that occurs in peripheral bypass grafts. Compare the 50% one patency in PTFE dialysis access grafts with an 88% five year patency for aortoiliac grafts and a 70 to 80% one year patency for femoro-popliteal grafts. Venous stenoses in the setting of dialysis access grafts also have a poorer response to angioplasty (40% three month survival if thrombosed and a 50% six month survival if not thrombosed) as compared to arterial stenoses. According to Kelly & col, the lack of effective therapies for VNH and venous stenosis in dialysis grafts such as PTFE dialysis grafts Is due to (a) a lack of appreciation of the fact that venous stenosis may be very different from the more common arterial stenosis at the graft-artery anastomosisand (b) the absence of a validated large animal model of VNH to test out novel interventions. Another reason for a lack of effective therapies could be related to the high prevalence of diabetes in dialysis patients leading to accelerated vascular responses to injury. Despite the magnitude of the problem and the enormity of the cost, there are currently no effective therapies for the prevention or treatment of venous neointimal hyperplasia in dialysis grafts.

Accordingly, there is a need for effective treatment and drug delivery systems for revascularization procedure, e.g. preventing and treating intimal thickening or restenosis that occurs after injury, e.g. vascular injury, including e.g. surgical injury, e.g. revascularization-induced injury, e.g. also in heart or other grafts, for a stabilization procedure of vulnerable plaques, or for the prevention or treatment of vascular access dysfunctions.

It is also an object of this invention to provide a drug-containing medical device which allows sustained delivery of the pharmaceutical or sufficient pharmaceutical activity at or near the coated surfaces of the devices.

Also, it is an object of the invention to provide medical devices with stabilized complexed drug coatings and methods for making such devices.

Additionally, it is an object of the invention to provide a drug-releasing coating stents or medical devices to allow the timed or prolonged application of the drug to body tissue. It Is a further object of the invention to provide methods for making a drug-releasing medical device, which permit timed-delivery or long-term delivery of a drug. Thus, there is a need for improved bio-compatible complexed drug coatings which enhance the biostability, abrasion-resistance, lubricity and bio-activity of the surface of implantable medical devices, especially complexed drug coatings which contain heat-sensitive biomolecules. In particular, there is a need for improved, cost efficient complexed drug coatings and devices, which have antithrombogenic and/or anti-restenosis and/or anti-inflammatory properties and for more efficient methods of providing same. The present invention is directed to meeting these and other needs.

Surprisingly, it has been found that N-{5-[4-(4-methyl-piperazino-methyl)-benzoylamido]-2-methylphenyl}-4-(3-pyridyl)-2-pyrimidine-amine or a pharmaceutically acceptable salt thereof (hereinafter COMPOUND I) can be suitably administered in the prevention or reduction of vascular access dysfunction that accompanies the insertion or repair of an indwelling shunt, fistula or catheter in a patient in need thereof.

COMPOUND I or a pharmaceutically acceptable salt thereof shows an unexpected high potency to prevent or eliminate vascular access dysfunction because of its unexpected multifunctional activity, and its activity on different aspects of vascular access dysfunction.

The prior art does not correspond or lead one to anticipate with any degree of certainty that the treatment with COMPOUND I would have a beneficial or therapeutically significant effect on the prevention or reduction of vascular access dysfunction that accompanies the insertion or repair of an indwelling shunt, fistula or catheter, such as a large bore catheter, into a vein in a mammal, particularly a human, in need thereof.

It has furthermore been found that COMPOUND I, optionally in conjunction with other active compounds, e.g. compounds having mTOR inhibiting properties or compounds having anti-inflammatory properties, have beneficial effects when locally applied to the lesions sites. It has particularly been found that COMPOUND I Is surprisingly well adapted for delivery especially controlled delivery from a catheter-based device (e.g. stents, indwelling shunt, fistula or catheter) or an intraluminal medical device. The pharmaceutically acceptable polymers do not alter or adversely impact the therapeutic properties of COMPOUND I. On the contrary, COMPOUND I, is particularly stable in any pharmaceutically acceptable polymers at body temperature and in human plasma, permitting an unexpected long storage in a coated stents, indwelling shunt, fistula or catheter.

COMPOUND I is particularly well adapted because it is easily secured onto the medical device by the polymer(s) (e.g. such as described herein) and the rate at which it is released from coating to the body tissue can be easily controlled. Furthermore, COMPOUND I coated stents permit long-term delivery of the drug. It is particularly worthwhile to control the bioeffectiveness of the COMPOUND I coated stents, indwelling shunt, fistula or catheter in order to obtain the same biological effect as a liquid dosage.

The preparation of N-{5-[4-(4-methyl-piperazino-methyl)-benzoylamido]-2-methylphenyl}-4-(3-pyridyl)-2-pyrimidine-amine or 4-(4-methylpiperazin-1-ylmethyl)-N-[4-methyl-3-(4-pyridin-3-yl)pyrimidin-2-ylamino)phenyl]-benzamide (hereinafter: COMPOUND I or “Imatinib” [International Non-proprietary Name]) and the use thereof, especially as an antiproliferative agent, are described in EP-A-0 564 409, which was published on 6 Oct. 1993, in U.S. Pat. No. 5,521,184 issued May 28, 1994 or in JP 2 706 682.

The term “4-(4-methylpiperazin-1-ylmethyl)-N-[4-methyl-3-(4-pyridin-3-yl)pyrimidin-2-yl-amino)phenyl]-benzamide” (hereinafter: COMPOUND I or “Imatinib” [International Non-proprietary Name]) includes the β-crystal form or a pharmaceutically acceptable salts thereof.

The preparation of COMPOUND I and the use thereof, especially as an anti-tumour agent, are described in Example 21 of European patent application EP-A-0 564 409, which was published on 6 Oct. 1993, and in equivalent applications and patents in numerous other countries, e.g. in U.S. Pat. No. 5,521,184 and in Japanese patent 2706682.

It will be understood that references to COMPOUND I meant to also include the pharmaceutically acceptable salts or β-crystal form thereof. COMPOUND I or a pharmaceutically acceptable salt or β-crystal form thereof may also be used in form of a hydrate or include other solvents used for crystallization. N-{5-[4-(4-methyl-piperazino-methyl)-benzoylamido]-2-methylphenyl}-4-(3-pyridyl)-2-pyrimidine-amine, is preferably used in the present invention in the form of its monomesylate salt. The β-crystal form or a pharmaceutically acceptable salt thereof are described in the European patent application No. 998 473.

The tyrosine kinase inhibitor COMPOUND I has recently shown promising results in the treatment of chronic myelogenous leukaemia (CML) and gastrointestinal stroma tumors (GIST).

COMPOUND I is a protein-tyrosine kinase inhibitor that is currently in clinical trials for the treatment of chronic myelogenous leukemia. COMPOUND I selectively inhibits the Abl and platelet-derived growth factor (PDGF) receptor tyrosine kinases in vitro and blocks cellular proliferation and tumor growth of Bcr-abl- or v-abl-expressing cells. COMPOUND I was further found to potently inhibit the kinase activity of the α- and β-PDGF receptors and the receptor for stem cell factor, but not the closely related c-Fms, Flt-3, Kdr, Flt-1, and Tek tyrosine kinases. Additionally, no inhibition of c-Met or nonreceptor tyrosine kinases such as Src and Jak-2 has been observed. In cell-based assays, COMPOUND I selectively inhibited PDGF and stem cell factor-mediated cellular signaling, including ligand-stimulated receptor autophosphorylation, inositol phosphate formation, and mitogen-activated protein kinase activation and proliferation. COMPOUND I has been shown to regulate the development of cardiac and aortic allograft arteriosclerosis as well as ordinary atherosclerosis in hypercholesterolemic rabbits. Thus, COMPOUND I may provide new strategies for prevention of these fibroproliferative vascular disorders.

These results expand the profile of COMPOUND I and suggest that in addition to chronic myelogenous leukemia, COMPOUND I may have clinical potential in the treatment of diseases that Involve abnormal activation of Kit (i.e. c-Kit), Abl or PDGF receptor tyrosine kinases.

According to the Invention, COMPOUND I may be applied as the sole active Ingredient or In conjunction with;
a) an Immunosuppressive agent, e.g. a calcineurin inhibitor, e.g. a cyclosporin, for example cyclosporin A, ISA tx 247 or FK506,
b) an EDG-receptor agonist having lymphocyte depleting properties, e.g. FTY720 (2-amino-2-[2-(4-octylphenyl)ethyl]propane-1,3-diol in free form or in a pharmaceutically acceptable salt form, e.g. the hydrochloride) or an analogue such as described in WO96/06068 or WO 98/45249, e.g. 2-amino-2-{2-[4-(1-oxo-5-phenylpentyl)phenyl]ethyl}propane-1,3-diol or 2-amino-4-(4-heptyloxyphenyl)-2-methyl-butanol in free form or in a pharmaceutically acceptable salt form,
c) an anti-inflammatory agent, e.g. a steroid, e.g. a corticosteroid, e.g. dexamethasone or prednisone, a NSAID, e.g. a cyclooxygenase inhibitor, e.g. a cox-2 inhibitor, e.g. celecoxib, rofecoxib, etoricoxib or valdecoxib, an ascomycin, e.g. ASM981 (or pimecrolimus), a cytokine inhibitor, e.g. a lymphokine inhibitor, e.g. an IL-1, -2 or -6 inhibitor, for example prainacasan or anakinra, or a TNF inhibitor, for instance Etanercept, or a chemokine inhibitor;
d) an anti-thrombotic or anti-coagulant agent, e.g. heparin or a glycoprotein IIb/IIIa Inhibitor, e.g. abciximab, eptifibatide or tirofibran;
e) an antiproliferative agent, e.g.
   a microtubule stabilizing or destabilizing agent Including but not limited to taxanes, e.g. taxol, paclitaxel or docetaxel, vinca alkaloids, e.g. vinblastine, especially vinblastine sulfate, vincristine especially vincristine sulfate, and vinorelbine, discodermolides or epothilones or a derivative thereof, e.g. epothilone B or a derivative thereof;
   a protein tyrosine kinase inhibitor, e.g. protein kinase C or PI(3) kinase inhibitor, for example staurosporin and related small molecules, e.g. UCN-01, BAY 43-9006, Bryostatin 1, Perifosine, Limofosine, midostaurin, CGP52421, RO318220, RO320432, GO 6976, Isis 3521, LY333531, LY379196, SU5416, SU6668, AG1296, etc. Midostaurin is a derivative of the naturally occurring alkaloid staurosporine with the chemical name (N-[(9S,10R,11R,13R)-2,3,10,11,12,13-hexahydro-10-methoxy-9-methyl-1-oxo-9,13-epoxy-1H,9H-diindolo[1,2,3-gh:3′,2′,1′-lm]pyrrolo[3,4-j][1,7]benzodiazonin-11-yl]-N-methylbenzamide), and has been specifically described in the European patent No. 0 296 110 published on Dec. 21, 1988, as well as in U.S. Pat. No. 5,093,330 published on Mar. 3, 1992, and Japanese Patent No. 2 708 047 all in the name of the applicant. Midostaurin was originally identified as an inhibitor of protein kinase C (PKC) (Meyer T, Regenass U, Fabbro D, et al: Int J Cancer 43: 851-856, 1989).
   a compound or antibody which Inhibits the PDGF receptor tyrosine kinase or a compound which binds to PDGF or reduces expression of the PDGF receptor e.g. a N-phenyl-2-pyrimidine-amine derivative, CT52923, RP-1776, GFB-111, a pyrrolo[3,4-c]-beta-carboline-dione, etc.;
   a compound or antibody which inhibits the EGF receptor tyrosine kinase or a compound which binds to EGF or reduces expression of the EGF receptor e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839, Iressa) and WO 95/03283 (e.g. compound ZM105180); e.g. trastuzumab (Herpetin^{R}), cetuximab, OSI-774, CI-1033, EKB-569, GW-2016, E1.1, E2.4, E2.5, E6.2, E6.4, E2.11, E6.3 or E7.6.3, retinoic acid, alpha-, gamma- or delta-tocopherol or alpha-, gamma- or delta-tocotrienol, or compounds affecting GRB2, IMC-C225; or
   a compound or antibody which inhibits the VEGF receptor tyrosine kinase or a VEGF receptor or a compound which binds to VEGF, e.g. proteins, small molecules or monoclonal antibodies generically and specifically disclosed in WO 98/35958, e.g. 1-(4-chloroanilino)-4-(4-pyridylmethyl)phthalazine or a pharmaceutically acceptable salt thereof, e.g. the succinate, or in WO 00/09495, WO 00/27820, WO 00/59509, WO 98/11223, WO 00/27819, WO 00/37502, WO 94/10202 and EP 0 769 947, those as described by M. Prewett et al in Cancer Research 59 (1999) 5209-5218, by F. Yuan et al in Proc. Natl. Acad. Sci. USA, vol. 93, pp. 14765-14770, Dec. 1996, by Z. Zhu et al in Cancer Res. 58,1998,3209-3214, by J. Mordenti et al in Toxicologic Pathology, Vol. 27, no. 1, pp 14-21, 1999, Angiostatin™, described by M. S. O'Reilly et al, Cell 79, 1994,315-328, Endostatin™, described by M. S. O'Reilly et al, Cell 88, 1997, 277-285, anthranilic acid amides, ZD4190; ZD6474, SU5416, SU6668 or anti-VEGF antibodies or anti-VEGF receptor antibodies, e.g. RhuMab;
f) a statin, e.g. having HMG-CoA reductase Inhibition activity, e.g. fluvastatin, lovastatin, simvastatin, pravastatin, atorvastatin, cerivastatin, pitavastatin, rosuvastatin or nivastatin;
g) a compound, protein, growth factor or compound stimulating growth factor production that will enhance endothelial regrowth of the luminal endothelium, e.g. FGF, IGF;
h) a matrix metalloproteinase inhibitor, e.g. batimistat, marimistat, trocade, CGS 27023, RS 130830 or AG3340;
k) a modulator (i.e. antagonists or agonists) of kinases, e.g. JNK, ERK1/2, MAPK or STAT;
l) a compound stimulating the release of (NO) or a NO donor, e.g. diazeniumdiolates, S-nitrosothiols, mesolonic oxatriazoles, isosorbide or a combination thereof, e.g. mononitrate and/or dinitrate;
m) a somatostatin analogue, e.g. octreotide, lanreotide, vapreotide or a cyclohexapeptide having somatostatin agonist properties, e.g. cyclo[4-(NH₂—C₂H₄—NH—CO—O)Pro-Phg-DTrp-Lys-Tyr(Bzl)-Phe]; or a modified GH analogue chemically linked to PEG, e.g. Pegvisomant;
n) an altosterone synthetase Inhibitor or aldosterone receptor blocker, e.g. eplerenone, or a compound inhibiting the renin-angiotensin system, e.g. a renin inhibitor, e.g. SPP100, an ACE inhibitor, e.g. captopril, enalapril, lisinopril, fosinopril, benazepril, quinapril, ramipril, imidapril, perindopril erbumine, trandolapril or moexipril, or an ACE receptor blocker, e.g. losartan, irbesartan, candesartan cilexetil, valsartan or olmesartan medoxomil;
o) mycophenolic acid or a salt thereof, e.g. sodium mycophenolate, or a prodrug thereof, e.g. mycophenolate mofetil.
p) a rapamycin derivative. Rapamycin is a known macrolide antibiotic produced by Streptomyces hygroscopicus, which inhibits mTOR. By rapamycin derivative having mTOR inhibiting properties is meant a substituted rapamycin, e.g. a 40-substituted-rapamycin or a 16-substituted rapamycin, or a 32-hydrogenated rapamycin. Representative rapamycin derivatives are e.g. 32-deoxorapamycin, 16-pent-2-ynyloxy-32-deoxorapamycin, 16-pent-2-ynyloxy-32(S or R)-dihydro-rapamycin, 16-pent-2-ynyloxy-32(S or R)-dihydro-40-O-(2-hydroxyethyl)-rapamycin, 40-[3-hydroxy-2-(hydroxymethyl)-2-methylpropanoate]-rapamycin (also called CCI779) or 40-epi-(tetrazolyl)-rapamycin (also called ABT578). A preferred compound is e.g. 40-0-(2-hydroxyethyl)-rapamycin disclosed in Example 8 in WO 94/09010, or 32-deoxorapamycin or 16-pent-2-ynyloxy-32(S)-dihydro-rapamycin as disclosed in WO 96/41807. Rapamycin derivatives may also include the so-called rapalogs, e.g. as disclosed in WO 98/02441 and WO01/14387, e.g. AP23573.

Are comprised also in the above list the pharmaceutically acceptable salts, the corresponding racemates, diastereoisomers, enantiomers, tautomers as well as the corresponding crystal modifications of above disclosed compounds where present, e.g. solvates, hydrates and polymorphs.

By antibody is meant monoclonal antibodies, polyclonal antibodies, multispecific antibodies formed from at least 2 intact antibodies, and antibodies fragments so long as they exhibit the desired biological activity.

According to the invention, COMPOUND I is preferably locally administered or delivered in conjunction with one or more co-agents selected from a), b), c), d), e), f), g), h), i), j), k), l), m), n), o), p), a cox-2 inhibitor, a cytokine inhibitor or a chemokine inhibitor, as defined above.

Hence, the present invention also relates to a method of treating a warm-blooded animal having a disease as mentioned herein, comprising administering to the animal a combination which comprises (a) N-{5-[4-(4-methyl-piperazino-methyl)-benzoylamido]-2-methylphenyl}-4-(3-pyridyl)-2-pyrimidine-amine and (b) one or more co-agents selected from a), b), c), d), e), f), g), h), i), j), k), l), m), n), o), p), a cox-2 inhibitor, a cytokine inhibitor or a chemokine inhibitor, as defined above, in a quantity which is jointly therapeutically effective against the disease and in which the compounds can also be present in the form of their pharmaceutically acceptable salts.

Furthermore, the present invention pertains to a combination, such as a combined preparation or a pharmaceutical composition, which comprises (a) N-{5-[4-(4-methyl-piperazino-methyl)-benzoylamido]-2-methylphenyl}-4-(3-pyridyl)-2-pyrimidine-amine and (b) one or more co-agents selected from a), b), c), d), e), f), g), h), i), j), k), l), m), n), o), p), a cox-2 inhibitor, a cytokine inhibitor or a chemokine Inhibitor, as defined above, wherein the active ingredients are present in each case in free form or in the form of a pharmaceutically acceptable salt, and optionally at least one pharmaceutically acceptable carrier; for simultaneous, separate or sequential use.

A combination which comprises (a) N-{5-[4-(4-methyl-piperazino-methyl)-benzoylamido]-2-methylphenyl}-4-(3-pyridyl)-2-pyrimidine-amine and (b) one or more co-agents selected from a), b), c), d), e), f), g), h), i), j), k), l), m), n), o), p), a cox-2 inhibitor, a cytokine inhibitor or a chemokine inhibitor, as defined above, in each case in free form or in the form of a pharmaceutically acceptable salt and optionally at least one pharmaceutically acceptable carrier, will be referred to hereinafter as a COMBINATION OF THE INVENTION.

The preferred co-agents within the meaning of the present Invention, are selected from a rapamycin derivative having mTOR inhibiting properties or rapamycin, an EDG-receptor agonist having lymphocyte depleting properties, a cox-2 inhibitor, pimecrolimus, a cytokine inhibitor, a chemokine inhibitor, an antiproliferative agent, a statin, a protein, growth factor or compound stimulating growth factor production that will enhance endothelial regrowth of the luminal endothelium, a matrix metalloproteinase inhibitor, a somatostatin analogue, an aldosterone synthetase inhibitor or aldosterone receptor blocker and a compound inhibiting the renin-angiotensin system. Most preferably active co-agents selected from a calcineurin inhibitor, mycophenolic acid, rapamycin and midostaurin or a salt thereof or prodrug thereof, each being releasably affixed to the drug delivery device or system.

Furthermore, the present invention pertains to a combination as described above, in a form especially adapted for coated delivery device or system as described herein (e.g. stent, catheter, . . . ). Preferably, in the form of a slow release pharmaceutical composition (controlled delivery).

All the more surprising is the experimental finding that in vivo the administration of a COMBINATION OF THE INVENTION, results not only in a beneficial effect, especially a synergistic therapeutic effect, e.g. with regard to slowing down, arresting or reversing the herein described diseases, but also in further surprising beneficial effects, e.g. less side-effects, an improved quality of life and a decreased mortality and morbidity, compared to a monotherapy applying only one of the pharmaceutically active ingredients used in the COMBINATION OF THE INVENTION. In particular, an increased up-take of the combination partner (b) in cells is observed, when applied in combination with combination partner (a).

The COMBINATION OF THE INVENTION can be a combined preparation or a pharmaceutical composition.

It is one objective of this invention to provide a pharmaceutical composition comprising a quantity, which is jointly therapeutically effective against a disease as described herein, comprising the COMBINATION OF THE INVENTION. In this composition, the combination partners (a) and (b) can be administered together, one after the other or separately in one combined unit dosage form or in two separate unit dosage forms. The unit dosage form may also be a fixed combination.

The pharmaceutical compositions for separate administration of the combination partners (a) and (b) and for the administration in a fixed combination, i.e. a single galenical compositions comprising at least two combination partners (a) and (b), according to the invention can be prepared in a manner known per se and are those suitable for enteral, such as oral or rectal, and parenteral administration to mammals (warm-blooded animals), including man, comprising a therapeutically effective amount of at least one pharmacologically active combination partner alone or in combination with one or more pharmaceutically acceptable carries, especially suitable for enteral or parenteral application.

Novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not Indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the COMBINATION OF THE INVENTION may be administered simultaneously or sequentially and in any order, and the components may be administered separately or as a fixed combination. For example, the method of delay of progression or treatment of a disease according to the Invention may comprise (i) administration of the combination partner (a) in free or pharmaceutically acceptable salt form and (ii) administration of a combination partner (b) in free or pharmaceutically acceptable salt form, simultaneously or sequentially in any order, in jointly therapeutically effective amounts, preferably in synergistically effective amounts, e.g. In daily dosages corresponding to the amounts described herein. The Individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients' availability to target sites.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b). The ratio of the total amounts of the combination partner (a) to the combination partner (b) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, a combined therapeutical effect in a non-effective dosage of one or both of the combination partners (a) and (b), and very preferably a strong synergism of the combination partners (a) and (b).

The present invention also provides the administration, local administration or delivery of COMPOUND I in conjunction with a calcineurin inhibitor, e.g. as disclosed above, a mTOR inhibitor agent e.g. rapamycin derivatives, e.g. 40-O-(2-hydroxyethyl)-rapamycin, an EDG-Receptor agonist, e.g. as disclosed above, a microtubule stabilizing or destabilizing agent, e.g. as disclosed above, a compound or antibody which inhibits the PDGF receptor tyrosine kinase or a compound which binds to PDGF or reduces expression of the PDGF receptor, e.g. as disclosed above, a compound or antibody which inhibits the EGF receptor tyrosine kinase or a compound which binds to EGF or reduces expression of the EGF receptor, e.g. as disclosed above, a compound or antibody which inhibits the VEGF receptor tyrosine kinase or a VEGF receptor or a compound which binds to VEGF, e.g. as disclosed above, an inhibitor of a modulator (i.e. antagonists or agonists) of kinases, e.g. as disclosed above.

In accordance with the particular findings of the present invention, there is provided;

1.1 A method for preventing or treating smooth muscle cell proliferation and migration in hollow tubes (e.g. catheter-based device), or increased cell proliferation or decreased apoptosis or increased matrix deposition in a mammal in need thereof, comprising local administration of a therapeutically effective amount of COMPOUND I, optionally in conjunction with one or more other active ingredients, e.g. as disclosed above.

1.2 A method for the treatment of intimal thickening in vessel walls comprising the controlled delivery from any catheter-based device (e.g. indwelling shunt, fistula or catheter) or intraluminal medical device of a therapeutically effective amount of a COMPOUND I, optionally in conjunction with one or more other active ingredients, e.g. as disclosed above. Preferably the treatment of intimal thickening in vessel walls is remodeling, hypertrophic remodeling, matrix deposition, fibrin deposit, neointima growth, stenosis, restenosis, e.g. following revascularization or neovascularization, and/or inflammation and/or thrombosis.

1.3 A method for the prevention or treatment of inflammatory disorders, e.g. T-cell induced Inflammation, in hollow tubes comprising the controlled delivery from any catheter-based device, intraluminal medical device or adventitial medical device of a therapeutically effective amount of COMPOUND I, optionally in conjunction with one or more other active co-agents, e.g. as disclosed above.

1.4 A method for stabilizing vulnerable plaques in blood vessels of a subject In need of such a stabilization comprising the controlled delivery from any catheter-based device, intraluminal medical device or adventitial medical device of a therapeutically effective amount of COMPOUND I, optionally in conjunction with one or more other active co-agents, e.g. as disclosed above.

1.5 A method as defined in 1.1 to 1.4 associated, simultaneously or sequentially, with the administration of a therapeutically effective amount of COMPOUND I. Preferably COMPOUND I, is administered orally.

Alternatively, a method as defined in 1.1 to 1.4 may be associated, simultaneously or sequentially, with the administration of a therapeutically effective amount of the co-agent.

1.6 A method for preventing or treating restenosis in diabetic patients comprising administering to said patients a therapeutically effective amount of COMPOUND I, optionally in conjunction with one or more other active co-agents, e.g. as disclosed above.

1.7 A method for preventing or treating restenosis (e.g. restenosis in diabetic patients, hypertensive patients, . . . ) comprising the controlled delivery from any catheter-based device, intraluminal medical device or adventitial medical device of a therapeutically effective amount of COMPOUND I, optionally in conjunction with one or more other active co-agents, e.g. as disclosed above.

1.8 A method comprising a combination of method steps as disclosed above under 1.6 and 1.7.

1.9 A method for the prevention or reduction of vascular access dysfunction in association with the Insertion or repair of an indwelling shunt, fistula or catheter, preferably a large bore catheter, into a vein or artery, or actual treatment, in a subject in need thereof, which comprises administering to the subject COMPOUND I, optionally in conjunction with one or more other active co-agents, e.g. as disclosed above, or a controlled delivery from a drug delivery medical device or system of a therapeutically effective amount of COMPOUND I, optionally in conjunction with one or more other active co-agents, e.g. as disclosed above.

Preferably the invention relates to the prevention or reduction of vascular access dysfunction in dialysis patients (e.g. hemodialysis).

1.10 A method for the stabilization or repair of arterial or venous aneurisms in a subject comprising the controlled delivery from any catheter-based device, intraluminal medical device or adventitial medical device of a therapeutically effective amount of COMPOUND I, optionally in conjunction with one or more other active co-agents, e.g. as disclosed above.

1.11 A method for the prevention or treatment of anastomic hyperplasia in a subject comprising the controlled delivery from any catheter-based device, intraluminal medical device or adventitial medical device of a therapeutically effective amount of COMPOUND I, optionally in conjunction with one or more other active co-agents, e.g. as disclosed above.

1.12 A method for the prevention or treatment of arterial, e.g. aortic, by-pass anastomosis in a subject comprising the controlled delivery from any catheter-based device, intraluminal medical device or adventitial medical device of a therapeutically effective amount of COMPOUND I, optionally in conjunction with one or more other active co-agents, e.g. as disclosed above.

1.13 A method as defined in 1.9 to 1.12 associated, simultaneously or sequentially, with the administration of a therapeutically effective amount of COMPOUND I. Preferably COMPOUND I, is administered orally.

Alternatively, a method as defined in 1.9 to 1.12 may be associated, simultaneously or sequentially, with the administration of a therapeutically effective amount of the co-agent.

2.1 A drug delivery device or system comprising a) a medical device adapted for local application or administration in hollow tubes, e.g. a catheter-based delivery device (e.g. indwelling shunt, fistula or catheter) or a medical device intraluminal or outside of hollow tubes such as an implant or a sheath placed within the adventitia, and b) a therapeutic dosage of COMPOUND I, optionally in conjunction with a therapeutic dosage of one or more other active ingredients, e.g. as disclosed above, each being releasably affixed to the catheter-based delivery device or medical device.

2.2 A device as defined herein for use in any method as defined under 1.1 to 1.12.

3.1 Use of COMPOUND I In any of the method as defined under 1.4, 1.6 or 1.9 optionally In conjunction with one or more other active co-agent, or in the manufacture of a medicament for use in any of the method as defined under 1.4, 1.6 or 1.9 optionally in conjunction with one or more other active co-agent.

3.2 Use of a COMPOUND I, optionally in combination with an active co-agent as defined herein, in the manufacture of a device as defined herein for use in any method as defined under 1.1 to 1.12.

3.3 Use of indwelling shunt, fistula or catheter coated by, impregnated with or incorporating COMPOUND I (i.e. being releasably affixed to the medical device) as described herein, for the manufacture of a medicament for the prevention or reduction of vascular access dysfunction in association with the insertion or repair of an indwelling shunt, fistula or catheter into a vein or artery, in a subject in need thereof.

4 A pharmaceutical composition for use in any method as defined under 1.4, 1.6 or 1.9 comprising COMPOUND I, together with one or more pharmaceutically acceptable diluents or carriers therefore.

Such a local delivery device or system can be used to reduce the herein mentioned vascular injuries e.g. stenosis, restenosis, or in-stent restenosis, as an adjunct to revascularization, bypass or grafting procedures performed in any vascular location Including coronary arteries, carotid arteries, renal arteries, peripheral arteries, cerebral arteries or any other arterial or venous location, to reduce anastomic stenosis or hyperplasia, including in the case of arterial-venous dialysis access with or without polytetrafluoroethylene or e.g. Gore-Tex grafting and with or without stenting, or in conjunction with any other heart or transplantation procedures, or congenital vascular Interventions. In a preferred embodiment, the present invention also provides a drug delivery system or device as disclosed above additionally comprising a source delivering a therapeutic dosage of a compound a rapamycin derivative having mTOR Inhibiting properties or rapamycin, EDG-receptor agonist having lymphocyte depleting properties, a cox-2 inhibitor, pimecrolimus, a cytokine inhibitor, a chemokine inhibitor, an antiproliferative agent, a statin, a protein, growth factor or compound stimulating growth factor production that will enhance endothelial re-growth of the luminal endothelium, a matrix metalloproteinase inhibitor, a somatostatin analogue, an aldosterone synthetase inhibitor or aldosterone receptor blocker and a compound inhibiting the renin-angiotensin system, or an antibody which inhibits the PDGF receptor tyrosine kinase or a compound which binds to PDGF or reduces expression of the PDGF receptor e.g. as disclosed above, a compound or antibody which Inhibits the EGF receptor tyrosine kinase or a compound which binds to EGF or reduces expression of the EGF receptor e.g. as disclosed above, a compound or antibody which inhibits the VEGF receptor tyrosine kinase or a VEGF receptor or a compound which binds to VEGF, each being releasably affixed to the catheter-based delivery device or medical device.

Reocclusion following stenting is due to both restenotic lesion formation within the stent boundaries and constrictive remodeling at both the proximal and distal margins of the local delivery device or system (e.g. stent). COMPOUND I is particularly useful, because it furthermore reduces the constrictive remodeling at both the proximal and distal margins of the local delivery device or system (e.g. stent). Many compounds (e.g. sirolimus) currently used do not significantly inhibit such constrictive remodeling (inhibition is solely significant for neointimal lesion size). Thus, COMPOUND I provides an unexpected advantage over currently used compounds and local delivery device or system as described herein coated by, impregnated with or incorporating COMPOUND I are particularly useful.

COMPOUND I or a pharmaceutically acceptable salt thereof will be referred to hereinafter as “drug”. The other active ingredients which may be used in conjunction with COMPOUND I as disclosed above, will be referred to hereinafter collectively as “adjunct”. Drug(s) shall mean drug or drug+adjunct.

The local administration preferably takes place at or near the vascular lesions sites.

The administration may be by one or more of the following routes: via catheter or other intravascular delivery system, intranasally, intrabronchially, interperitoneally or eosophagal. Hollow tubes include circulatory system vessels such as blood vessels (arteries or veins), tissue lumen, lymphatic pathways, digestive tract including alimentary canal, respiratory tract, excretory system tubes, reproductive system tubes and ducts, body cavity tubes, etc. Local administration or application of the drug(s) affords concentrated delivery of said drug(s), achieving tissue levels in target tissues not otherwise obtainable through other administration route. Additionally local administration or application may reduce the risk of remote or systemic toxicity. Preferably the smooth muscle cell proliferation or migration is inhibited or reduced according to the invention immediately proximal or distal to the locally treated or stented area.

Means for local drug(s) delivery to hollow tubes can be by physical delivery of the drug(s) either. Internally or externally to the hollow tube. Local drug(s) delivery includes catheter delivery systems, local injection devices or systems or indwelling devices. Such devices or systems would include, but not be limited to, indwelling shunt, fistula, catheter, stents, endolumenal sleeves, stent-grafts, liposomes, controlled release matrices, polymeric endoluminal paving, or other endovascular devices, embolic delivery particles, cell targeting such as affinity based delivery, internal patches around the hollow tube, external patches around the hollow tube, hollow tube cuff, external paving, external stent sleeves, and the like. See, Eccleston et al. (1995) Interventional Cardiology Monitor 1:33-40-41 and Slepian, N.J. (1996) Intervente. Cardiol. 1:103-116, or Regar E, Sianos G, Serruys P W. Stent development and local drug delivery. Br Med Bull 2001,59:227-48 which disclosures are herein incorporated by reference. Preferably the delivery device or system fulfils pharmacological, pharmacokinetic and mechanical requirements. Preferably it also is suitable for sterilization.

The stent according to the invention can be any stent, including self-expanding stent, or a stent that is radially expandable by inflating a balloon or expanded by an expansion member, or a stent that is expanded by the use of radio frequency which provides heat to cause the stent to change its size.

Delivery or application of the drug(s) can occur using indwelling shunt, fistula, stents or sleeves or sheathes. An stent composed of or coated with a polymer or other biocompatible materials, e.g. porous ceramic, e.g. nanoporous ceramic, into which the drug(s) has been Impregnated or incorporated can be used. Such stents can be biodegradable or can be made of metal or alloy, e.g. Ni and Ti, or another stable substance when intented for permanent use. The drug(s) may also be entrapped into the metal of the stent or graft body which has been modified to contain micropores or channels. Also lumenal and/or ablumenal coating or external sleeve made of polymer or other biocompatible materials, e.g. as disclosed above, that contain the drug(s) can also be used for local delivery.

By “biocompatible” Is meant a material which elicits no or minimal negative tissue reaction including e.g. thrombus formation and/or inflammation.

Stents may commonly used as a tubular structure left inside the lumen of a duct or vessel to relieve an obstruction. They may be inserted into the duct lumen in a non-expanded form and are then expanded autonomously (self-expanding stents) or with the aid of a second device in situ, e.g. a catheter-mounted angioplasty balloon which is inflated within the stenosed vessel or body passageway in order to disrupt the obstructions associated with the wall components of the vessel and to obtain an enlarged lumen. Alternatively, stents being easily deformed at lower temperature to be Inserted In the hollow tubes may be used: after deployment at site, such stents recover their original shape and exert a retentive and gentle force on the internal wall of the hollow tubes, e.g. of the esophagus or trachea.

For example, the drug(s) may be incorporated into or affixed to the stent (or to indwelling shunt, fistula or catheter) in a number of ways and utilizing any biocompatible materials; it may be incorporated into e.g. a polymer or a polymeric matrix and sprayed onto the outer surface of the stent. A mixture of the drug(s) and the polymeric material may be prepared in a solvent or a mixture of solvents and applied to the surfaces of the stents also by dip-coating, brush coating and/or dip/spin coating, the solvent (s) being allowed to evaporate to leave a film with entrapped drug(s). In the case of stents where the drug(s) is delivered from micropores, struts or channels, a solution of a polymer may additionally be applied as an outlayer to control the drug(s) release; alternatively, the drug may be comprised in the micropores, struts or channels and the adjunct may be incorporated in the outlayer, or vice versa. The drug may also be affixed in an inner layer of the stent (or of the indwelling shunt, fistula or catheter) and the adjunct in an outer layer, or vice versa. The drug(s) may also be attached by a covalent bond, e.g. esters, amides or anhydrides, to the stent (or of the indwelling shunt, fistula or catheter) surface, involving chemical derivatization. The drug(s) may also be incorporated into a biocompatible porous ceramic coating, e.g. a nanoporous ceramic coating. The medical device of the invention is configured to release the active co-agent concurrent with or subsequent to the release of the active agent.

Examples of polymeric materials include hydrophilic, hydrophobic or biocompatible biodegradable materials, e.g. polycarboxylic acids; cellulosic polymers; starch; collagen; hyaluronic acid; gelatin; lactone-based polyesters or copolyesters, e.g. polylactide; polyglycolide; polylactide-glycolide; polycaprolactone; polycaprolactone-glycolide; poly(hydroxybutyrate); poly(hydroxyvalerate); polyhydroxy(butyrate-co-valerate); polyglycolide-co-trimethylene carbonate; poly(diaxanone); polyorthoesters; polyanhydrides; polyaminoacids; polysaccharides; polyphospoeters; polyphosphoester-urethane; polycyanoacrylates; polyphosphazenes; poly(ether-ester) copolymers, e.g. PEO-PLLA, fibrin; fibrinogen; or mixtures thereof; and biocompatible non-degrading materials, e.g. polyurethane; polyolefins; polyesters; polyamides; polycaprolactame; polyimide; polyvinyl chloride; polyvinyl methyl ether; polyvinyl alcohol or vinyl alcohoVolefin copolymers, e.g. vinyl alcohol/ethylene copolymers; polyacrylonitrile; polystyrene copolymers of vinyl monomers with olefins, e.g. styrene acrylonitrile copolymers, ethylene methyl methacrylate copolymers; polydimethylsiloxane; poly(ethylene-vinylacetate); acrylate based polymers or coplymers, e.g. polybutylmethacrylate, poly(hydroxyethyl methylmethacrylate); polyvinyl pyrrolidinone; fluorinated polymers such as polytetrafluoethylene; cellulose esters e.g. cellulose acetate, cellulose nitrate or cellulose proplonate; or mixtures thereof.

When a polymeric matrix is used, it may comprise 2 layers, e.g. a base layer in which the drug(s) is/are incorporated, e.g. ethylene-co-vinylacetate and polybutylmethacrylate, and a top coat, e.g. polybutylmethacrylate, which is drug(s)-free and acts as a diffusion-control of the drug(s). Alternatively, the drug may be comprised in the base layer and the adjunct may be incorporated in the outlayer, or vice versa. Total thickness of the polymeric matrix may be from about 1 to 20μ or greater.

According to the method of the invention or in the device or system of the invention, the drug(s) may elute passively, actively or under activation, e.g. light-activation.

The drug(s) elutes from the polymeric material or the stent, indwelling shunt, fistula or catheter, over time and enters the surrounding tissue, e.g. up to ca. 1 month to 1 year. The local delivery according to the present invention allows for high concentration of the drug(s) at the disease site with low concentration of circulating compound. The amount of drug(s) used for local delivery applications will vary depending on the compounds used, the condition to be treated and the desired effect. For purposes of the invention, a therapeutically effective amount will be administered; for example, the drug delivery device or system is configured to release the active agent and/or the active co-agent at a rate of 0.001 to 800 μg/day, preferably 0.001 to 200 μg/day. By therapeutically effective amount Is intended an amount sufficient to inhibit cellular proliferation and resulting in the prevention and treatment of the disease state. Specifically, for the prevention or treatment of vascular problems e.g. after revascularization, or antitumor treatment, local delivery may require less compound than systemic administration.

The present invention relates to a method for the prevention or reduction of vascular access dysfunction in association with the insertion or repair of an indwelling shunt, fistula or catheter, preferably a large bore catheter, or actual treatment, into a vein in a mammal, particularly a human, which comprises administering to the subject N-{5-[4-(4-methyl-piperazino-methyl)-benzoylamido]-2-methylphenyl}-4-(3-pyridyl)-2-pyrimidine-amine (COMPOUND I) or a pharmaceutically acceptable salt thereof in a dose of from about 0.1 mg to 2400 mg, preferably from about 10 mg to 1000 mg, most preferably from about 10 mg to 600 mg.

The present invention further relates to a method for the prevention or reduction of vascular access dysfunction in association with the insertion or repair of an indwelling shunt, fistula or catheter, preferably a large bore catheter, or actual treatment, into a vein in a mammal, particularly a human, which comprises administering to the subject N-{5-[4-(4-methyl-piperazino-methyl)-benzoylamido]-2-methylphenyl}-4-(3-pyridyl)-2-pyrimidine-amine (COMPOUND I) or a pharmaceutically acceptable salt thereof in a daily dose of from about 0.1 mg to 2400 mg, preferably from about 10 mg to 1000 mg, most preferably from about 10 mg to 600 mg for a treatment period of at least one week, preferably at least two weeks, in association with the insertion or repair of an indwelling shunt, fistula or catheter, preferably a large bore catheter, or actual treatment.

A preferred daily dosage amount of COMPOUND I for use in the present invention is about 0.1 mg to 2400 mg, preferably from about 10 mg to 1000 mg, most preferably from about 10 mg to 600 mg. A more preferred daily dosage amount of COMPOUND I for use in the present invention is from about 50 mg to about 600 mg. Particularly preferred Is a daily dosage amount of from about 100 mg to 200 mg of COMPOUND I for use in the present invention.

A contemplated treatment period for use In the present invention is about 85 days in association with the insertion or repair of an indwelling shunt, fistula or catheter, or actual treatment. A contemplated treatment period for use in the present invention is about 70 days in association with the insertion or repair of an indwelling shunt, fistula or catheter, or actual treatment. An additional contemplated treatment period for use in the present invention is about 50 days in association with the insertion or repair of an indwelling shunt, fistula or catheter, or actual treatment. A preferred treatment period for use in the present invention is about 28 days in association with the insertion or repair of an indwelling shunt, fistula or catheter, or actual treatment. An additional contemplated treatment period for use in the present invention is 14 days in association with the insertion or repair of an indwelling shunt, fistula or catheter, or actual treatment.

A preferred method of use in the current invention is a method for preventing or reducing vascular thrombosis and/or fistula failure and/or shunt failure and/or vascular access clotting and/or stenosis and/or restenosis and/or the need for declotting an indwelling access clotting shunt, fistula or catheter associated with insertion or repair of the indwelling shunt, fistula or catheter, or actual treatment, in dialysis patients.

A preferred method of use in the current invention is a method for preventing or reducing vascular thrombosis and/or fistula failure and/or shunt failure and/or vascular access clotting and/or stenosis and/or restenosis and/or the need for declotting an indwelling vascular access shunt, fistula or catheter associated with insertion or repair of the indwelling shunt, fistula or catheter, or actual treatment, in cancer patients.

A preferred method of use in the current invention is a method for preventing or reducing vascular thrombosis and/or fistula failure and/or shunt failure and/or vascular access clotting and/or stenosis and/or restenosis and/or the need for declotting an indwelling vascular access shunt, fistula or catheter associated with insertion or repair of the indwelling shunt, fistula or catheter, or actual treatment, in total parenteral nutrition (TPN) patients.

A preferred method of use in the current invention is a method for preventing or reducing vascular thrombosis and/or fistula failure and/or shunt failure and/or vascular access clotting and/or stenosis and/or restenosis and/or the need for declotting an indwelling vascular access shunt, fistula or catheter associated with insertion or repair of the indwelling shunt, fistula or catheter, or actual treatment, in patients underlying condition that may predispose to accelerated or retarded vascular response to injury e.g. in dialysis patients.

Finally, this invention also relates to the use of indwelling shunt, fistula or catheter coated by COMPOUND I (i.e. being releasably affixed to the medical device) as described herein, for the manufacture of a medicament for the prevention or reduction of vascular access dysfunction in association with the insertion or repair of an indwelling shunt, fistula or catheter into a vein, in a mammal in need thereof.

By the phrase “prevention or reduction of vascular access dysfunction in association with the insertion or repair of an indwelling shunt, fistula or catheter” as used herein, is meant that the Incidence of vascular thrombosis and/or fistula failure and/or shunt failure and/or vascular access clotting and/or stenosis and/or restenosis and/or the need for declotting an indwelling vascular access shunt, fistula or catheter in COMPOUND I treated patients collected over the observation period are prevented or reduced in comparison to untreated patients.

By the phrase “in association with the insertion or repair of an indwelling shunt, fistula or catheter” as used herein, is meant that the treatment with COMPOUND I can commence Immediately, for example within 4 to 8 hours, after insertion or repair of the indwelling shunt, fistula or catheter, or actual treatment, such as dialysis treatment; within a few days, for example about 7 days, preferably about 1 or 2 days, after insertion or repair of the Indwelling shunt, fistula or catheter, or actual treatment, such as dialysis treatment; or for a period of days, for example about 30 days, preferably about 14 days, preferably about 7 days, prior to insertion or repair of the indwelling shunt, fistula or catheter, or actual treatment, such as dialysis treatment. Also contemplated within the phrase “in association with the insertion or repair of an indwelling shunt, fistula or catheter” is a dosing protocol In which a dose or several doses, are skipped, for example in the morning of or on the day of insertion, repair or treatment. Also contemplated within the phrase “in association with the insertion or repair of an indwelling shunt, fistula or catheter” is a dosing protocol in which a day of drug treatment or several days of drug treatment, are skipped.

By the term “treatment” and derivatives thereof, is meant prophylactic and therapeutic therapy.

Included in term “treatment”, when used herein to refer surgical procedures, are procedures selected from access surgery, placement of fistula or shunt, catheter insertion, actual disease treatment, such as dialysis treatment, and declotting of an access shunt, fistula or catheter. Further, treatment for insertion access also includes repair/revision of the access. For example, a patient experiencing a failure in a dialysis access shunt will have the access repaired, for instance, by angioplasty.

By the term “collected over the observation period” as used herein, means a period of up to or about 12 months, preferably 12 months.

COMPOUND I or a pharmaceutically acceptable salt thereof, shows an unexpected high potency to prevent or eliminate vascular access dysfunction because of its unexpected multifunctional activity, and its activity on different aspects of vascular access dysfunction such as the narrowing of the lumen, smooth muscle cells proliferation and migration, accumulation of extracellular matrix, intimal thickening, angiogenesis within the neointima and adventitia, leukocyte recruitment, impairment of lymphocyte trafficking, activation of macrophage, macrophage cell layer lining the PTFE graft material, activation of cytokines and cell growth stimulating factors, venous neointimal hyperplasia (VNH), thrombosis, stenosis (e.g. at the arterial or veinal anastomosis), hemodialysis catheter-related infection, bile congestion, inflammation, and eventually occlusion in the biliary tree, vascular wall hypertrophy, hypertrophic remodeling (Mann M J. (Curr Cardiol Rep 2000 January;2(1):29-33)).

The invention relates especially to such method wherein a daily dose of 0.1 mg to 1000 mg, preferably from about 10 mg to 600 mg, most preferably from about 100 mg to 200 mg of COMPOUND I mesylate is administered.

Without further elaboration, it is believed that one skilled in the art can, using the preceding description, utilize the present invention to its fullest extent. The following examples are, therefore, to be construed as merely illustrative and not a limitation of the scope of the present invention in any way.

Preferred combinations are those comprising COMPOUND I in conjunction or association with a compound having antiproliferative properties, e.g. taxol, paclitaxel, docetaxel, an epothilone, a tyrosine kinase inhibitor, a VEGF receptor tyrosine kinase inhibitor, a VEGF receptor inhibitor, a compound binding to VEGF, a mTOR inhibitor agent e.g. rapamycin derivatives, e.g. 40-O-(2-hydroxyethyl)-rapamycin, a compound having anti-inflammatory properties, e.g. a steroid, a cyclooxygenase inhibitor, combination of COMPOUND I with a compound having anti-inflammatory properties has particularly beneficial effects when used in the treatment or prevention of restenosis in diabetic patients.

Utility of the drug(s) may be demonstrated in animal test methods as well as In clinic, for example in accordance with the methods hereinafter described.

### EXAMPLE 1

### Inhibition of Late Neointimal Lesion Formation in the 28 Day Rat Carotid Artery Balloon Injury Model

Numerous compounds have been shown to inhibit intimal lesion formation at 2 weeks in the rat ballooned carotid model, while only few compounds prove effective at 4 weeks. Compounds of formula I are tested in the following rat model.

Rats are dosed orally with placebo or a compound of formula 1. Daily dosing starts 3 days prior to surgery and continues for 31 days. Rat carotid arteries are balloon injured using a method described by Clowes et al., Lab. Invest. 1983;49;208-215. Following sacrifice at 28 days post-balloon Injury, carotid arteries are removed and processed for histologic and morphometric evaluation. In this assay COMPOUND I, significantly reduce neointimal lesion formation at 28 days following balloon Injury when administered at a dose of from 0.2 to 3.5 mg preferably 0.5 to 2.0 mg/kg. For example COMPOUND I administered at 0.5, 1.0, and 2.0 mg/kg, the percent inhibition is similar at all three doses: inhibition is 17% at the lowest dose (0.5 mg/kg) and 37% at the highest dose (2.0 mg/kg). COMPOUND I have the beneficial effect to inhibit lesions at 4 weeks post-ballooning.

### EXAMPLE 2

### Inhibition of Restenosis (e.g. In-Stent Restenosis) at 28 Days in the Rabbit Iliac Stent Model

A combined angioplasty and stenting procedure is performed in New Zealand White rabbit iliac arteries. Iliac artery balloon injury is performed by inflating a 3.0×9.0 mm angioplasty balloon in the mid-portion of the artery followed by “pull-back” of the catheter for 1 balloon length. Balloon injury is repeated 2 times, and a 3.0×12 mm stent is deployed at 6 atm for 30 seconds in the iliac artery. Balloon injury and stent placement is then performed on the contralateral iliac artery in the same manner. A post-stent deployment angiogram is performed. All animals receive oral aspirin 40 mg/day daily as anti-platelet therapy and are fed standard low-cholesterol rabbit chow. Twenty-eight days after stenting, animals are anesthetized and euthanized and the arterial tree is perfused at 100 mmHg with lactated Ringer's for several minutes, then perfused with 10% formalin at 100 mmHg for 15 minutes. The vascular section between the distal aorta and the proximal femoral arteries is excised and cleaned of periadventitial tissue. The stented section of artery is embedded in plastic and sections are taken from the proximal, middle, and distal portions of each stent. All sections are stained with hematoxylin-eosin and Movat pentachrome stains. Computerized planimetry is performed to determine the area of the internal elastic lamina (IEL), external elastic lamina (EEL) and lumen. The neointima and neointimal thickness is measured both at and between the stent struts. The vessel area is measured as the area within the EEL. Data are expressed as mean±SEM. Statistical analysis of the histologic data is accomplished using analysis of variance (ANOVA) due to the fact that two stented arteries are measured per animal with a mean generated per animal. A P<0.05 is considered statistically significant.

COMPOUND is administered orally by gavage at 30 mg/kg once daily from 3 days prior to stenting until day 27 post-stenting. In this model, the treatment with the compounds of formula I results in a marked reduction In the extent of restenotic lesion formation compared with placebo treatment: for example, the treatment with COMPOUND I produces a significant reduction in average neointimal thickness (29% reduction; P<0.0001), neointimal area (17% reduction P<0.04), and percent arterial stenosis (17% reduction P<0.0002). Treatment with COMPOUND I did not result in differences in EEL area compared with control, Indicating that treatment was not associated with either constrictive remodeling or aneurysmal-type arterial expansion. There is extensive neointimal formation in placebo-treated animals at 28 days, with the lesions consisting of abundant smooth muscle cells in proteoglycan/collagen matrix and apparent full endothelial healing. In arterial segments from the animals treated with COMPOUND I, the Intima is well healed, characterized by a compact neointima consisting of smooth muscle cells and endothelium fully covering the lumen surface both over stent struts and between struts.

COMPOUND I suppresses in-stent neointimal growth and remodeling (e.g. hypertrophic remodeling), reduced fibrin deposit, and is associated with neointimal and endothelial healing in rabbit iliac arteries. Thus, COMPOUND I is useful as a stent coating-elutant and/or as an oral adjunct to stents eluting this or other active co-agents.

The following example is illustrative of the invention without limitating it.

### EXAMPLE 3

The stent is manufactured from medical 316LS stainless steel and is composed of a series of cylindrically oriented rings aligned along a common longitudinal axis. Each ring consists of 3 connecting bars and 6 expanding elements. The stent is premounted on a delivery system. The active agent, e.g. COMPOUND 1 (0.50 mg/ml) optionally together with 2,6-di-tert-butyl-4-methylphenol (0.001 mg/ml), is incorporated into a polymer matrix based on a semi-crystalline ethylene-vinyl alcohol copolymer. The stent is coated with this matrix.

### EXAMPLE 4

A stent is weighed and then mounted for coating. While the stent is rotating, a solution of polylactide glycolide, 0.70 mg/ml of COMPOUND I, 0.0015 mg/ml and 1 mg/ml tyrosine kinase inhibitor dissolved in a mixture of methanol and tetrahydrofuran, is sprayed onto it. The coated stent is removed from the spray and allowed to air-dry. After a final weighing the amount of coating on the stent is determined.

### EXAMPLE 5

COMPOUND I stability in pharmaceutically acceptable polymers at body temperature and COMPOUND I release from polymer coatings.

Four 2 cm pieces of coated stents as described above are placed into 100 mL of phosphate buffer solution (PBS) having a pH of 7.4. Another 4 pieces from each series are placed into 100 mL of polyethylene glycol (PEG)/water solution (40/60 v/v, MW of PEG=400). The stent pieces are incubated at 37° C. in a shaker. The buffer and PEG solutions are changed daily and different assays are performed on the solution to determine the released COMPOUND I concentrations. Such assays can show a stable COMPOUND I release from coated stents for more than 45 days. By the term “stable COMPOUND I release” we mean less than 10% of variation of the drug release rate. Controlled release techniques used by the person skilled in the art allow an unexpected easy adaptation of the required COMPOUND I release rate. Thus, by selecting appropriate amounts of reactants in the coating mixture it is possible to easily control the bioeffectiveness of the COMPOUND I coated stents. Depending on the kind of coating technology used, the drug may be eluted from coating passively, actively or by light activation.

Release of COMPOUND I in plasma can also be studied. 1 cm pieces of a coated stent are put Into 1 mL of citrated human plasma (from Helena Labs.), which is in lyophilized form and is reconstituted by adding 1 mL of sterile deionized water. Three sets of stent plasma solutions are incubated at 37° C. and the plasma is changed daily. In a separate study, it was found that COMPOUND I in human plasma was stable at 37° C. for 72 hours. PDGF-stimulated receptor tyrosine kinase assay is performed on the last piece of each sample to determine the COMPOUND I activity. The inhibition of PDGF-stimulated receptor tyrosine kinase activity in vitro is measured in PDGF receptor immunocomplexes of BALB/c 3T3 cells, analogously to the method described by E. Andrejauskas-Buchdunger and U. Regenass in Cancer Research 52, 5353-5358 (1992). Such assays can show that the activity of COMPOUND I released from stent after 45 days is still 91% of that of the normal activity of COMPOUND I. In the same assay, free COMPOUND I shows a strong decrease of its activity day after day. These assays can prove the unexpected high stability of COMPOUND I in polymer coatings.

### EXAMPLE 6

### Examples of Synergic Combinations

Further experiments similar to that of example 1 revealed synergic combinations when COMPOUND I is used in conjunction with several agents as mentioned herein.

Data points just spanning the IC50 of the agents alone or in combination are entered into the CalcuSyn program (CalcuSyn, Biosoft, Cambridge UK). This program calculates a non-exclusive combination index (CI), whose value is indicative of the interaction of the two compounds, where CI˜1 represents nearly additive effects; 0.85-0.9 Indicates a slight synergism and a value below 0.85 indicates synergy.

A C.I. of 0.3±0.03 was obtained for the combination of STI571 and Taxol® and a C.I. of 0.4±0.04 for the combination of STI571 and doxorubicin. Slight synergism or synergy can be observed with Taxol, doxorubicin, Vinblastine and several other compounds as disclosed above. The combinations especially show a synergistic therapeutic effect, e.g. with regard to slowing down, arresting or reversing arteriosclerosis, thrombosis, vascular access dysfunction, restenosis and/or inflammation diseases, but also In further surprising beneficial effects, e.g. allowing for less side-effects, an improved quality of life and a decreased mortality and morbidity, compared to a monotherapy applying only one of the pharmaceutically active ingredients used in the combination.

### EXAMPLE 7

Efficacy of the invented method for the prevention or reduction of vascular access dysfunction in association with the insertion of an indwelling catheter into the vein of a patient is demonstrated by the following.

One hundred fifty prospective dialysis patients, who undergo successful insertion of an indwelling, large bore catheter, into a vein are selected for study. These patients are divided into two groups, and both groups do not differ significantly with sex, distribution of vascular condition or condition of lesions after insertion. One group (about 50 patients) receives COMPOUND I in a daily dose of 400 mg (hereinafter identified as group 1), and another group (about 100 patients) does not receive COMPOUND I (hereinafter identified as group H). In addition, patients may also be given a calcium antagonist, nitrates, anti-platelet agents, ACEi angiotensin converting enzyme inhibitors, ARBs agiotensin receptor blockers, or statins. These drugs are administered for 3 consecutive months following catheter insertion.

The comparative clinical data collected over the observation period of 6 months demonstrate the efficacy of 3 month COMPOUND I treatment for the prevention or reduction of vascular access dysfunction in patients after catheter insertion.

### EXAMPLE 8

Efficacy of the invented method for the prevention or reduction of vascular access dysfunction in association with the insertion of an indwelling catheter Into the vein of a patient is demonstrated by the methodology as described by Dr. Burnett S. Kelly and Col., (Kidney International Volume 62; Issue 6; Page 2272—December 2002) which is Incorporated into the present application by reference.

1. A pharmaceutical composition for stabilizing vulnerable plaques in blood vessels of a subject in need of such a stabilization, for preventing or treating restenosis in diabetic patients, or for the prevention or reduction of vascular access dysfunction in association with the insertion or repair of an indwelling shunt, fistula or catheter in a subject in need thereof, comprising N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, together with one or more pharmaceutically acceptable diluents or carriers therefore.

2. Use of N-{5-[4-(4-methyl-piperazino-methyl)-benzoylamido]-2-methylphenyl}4-(3-pyridyl)-2-pyrimidine-amine or a pharmaceutically acceptable salt or crystal form thereof, for the manufacture of a pharmaceutical for stabilizing vulnerable plaques in blood vessels of a subject in need of such a stabilization, for preventing or treating restenosis in diabetic patients, or for the prevention or reduction of vascular access dysfunction in association with the insertion or repair of an indwelling shunt, fistula or catheter in a subject in need thereof.

3. A method for the prevention or reduction of vascular access dysfunction in association with the insertion or repair of an indwelling shunt, fistula or catheter into a vein or artery, or actual treatment, in a mammal in need thereof, which comprises administering to the subject an effective amount of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof.

4. Use according to claim 1, for use in conjunction with one or more active co-agents.

5. Use according to claim 1, for use in dialysis patients.

6. Use according to claim 1, wherein a methanesulfonate salt of 4-(4-methylpiperazin-1-ylmethyl)-N-[4-methyl-3-(4-pyridin-3-yl)pyrimidin-2-ylamino)phenyl]-benzamide is administered.

7. Use according to claim 1, wherein 4-(4-methylpiperazin-1-ylmethyl)-N-[4-methyl-3-(4-pyridin-3-yl)pyrimidin-2-ylamino)phenyl]-benzamide or a pharmaceutically acceptable salt or crystal form thereof is administered in a daily dose of 10 mg to 1000 mg.

8. Use according to claim 1, wherein the treatment period commences about 7 days prior to access placement.

9. Use according to claim 1, wherein the vascular access dysfunction is selected from vascular access clotting, vascular thrombosis or restenosis.

10. Use according to claim 1, wherein the vascular access dysfunction is the need for an unclotting procedure.

11. Use according to claim 1, wherein the dosage is administered orally.

12. Use according to claim 1, wherein the subject is selected from a dialysis patient, a cancer patient or a patient receiving total parenteral nutrition.

13. A drug delivery device or system comprising i) a medical device adapted for local application or administration in hollow tubes and ii) a therapeutic dosage of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with a therapeutic dosage of one or more active co-agents selected from a rapamycin derivative having mTOR inhibiting properties or rapamycin, an EDG-receptor agonist having lymphocyte depleting properties, a cox-2 inhibitor, pimecrolimus, a cytokine inhibitor, a chemokine inhibitor, an antiproliferative agent, a statin, a protein, growth factor or compound stimulating growth factor production that will enhance endothelial regrowth of the luminal endothelium, a matrix metalloproteinase inhibitor, a somatostatin analogue, an aldosterone synthetase inhibitor or aldosterone receptor blocker and a compound inhibiting the renin-angiotensin system, each being releasably affixed to the drug delivery device or system.

14. A drug delivery device or system comprising i) a medical device adapted for local application or administration in hollow tubes and ii) a therapeutic dosage of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with a therapeutic dosage of one or more active co-agents selected from a calcineurin inhibitor, mycophenolic acid, 40-O-(2-hydroxyethyl)-rapamycin, rapamycin and midostaurin or a salt thereof or prodrug thereof,
each being releasably affixed to the drug delivery device or system.

15. A drug delivery device or system according to claim 13, for preventing or treating smooth muscle cell proliferation and migration in hollow tubes, or increased cell proliferation or decreased apoptosis or increased matrix deposition in a subject in need thereof.

16. A drug delivery device or system according to claim, for stabilizing vulnerable plaques in blood vessels, for preventing or treating restenosis, restenosis in diabetic patients or for the prevention or reduction of vascular access dysfunction in association with the insertion or repair of an indwelling shunt, fistula or catheter in a patient in need thereof.

17. A drug delivery device or system comprising i) a medical device adapted for local application or administration in hollow tubes and ii) a therapeutic dosage of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with a therapeutic dosage of one or more active co-agents, each being releasably affixed to the catheter-based delivery device or system, for use in stabilizing vulnerable plaques in blood vessels, for preventing or treating restenosis, restenosis in diabetic patients or for the prevention or reduction of vascular access dysfunction in association with the insertion or repair of an indwelling shunt, fistula or catheter in a patient in need thereof.

18. A method for preventing or treating smooth muscle cell proliferation and migration in hollow tubes, or increased cell proliferation or decreased apoptosis or increased matrix deposition in a subject in need thereof, comprising local administration of a therapeutically effective amount of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with a therapeutic dosage of one or more active co-agents selected from a rapamycin derivative having mTOR inhibiting properties or rapamycin, an EDG-receptor agonist having lymphocyte depleting properties, a cox-2 inhibitor, pimecrolimus, a cytokine inhibitor, a chemokine inhibitor, an antiproliferative agent, a statin, a protein, growth factor or compound stimulating growth factor production that will enhance endothelial regrowth of the luminal endothelium, a matrix metalloproteinase inhibitor, a somatostatin analogue, an aldosterone synthetase inhibitor or aldosterone receptor blocker and a compound inhibiting the renin-angiotensin system.

19. A method for stabilizing vulnerable plaques in blood vessels of a subject in need of such a stabilization comprising the controlled delivery from a drug delivery device or system of a therapeutically effective amount of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with a therapeutic dosage of one or more active co-agents.

20. A method for preventing or treating restenosis in diabetic patients comprising administering to said patients a therapeutically effective amount of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with a therapeutic dosage of one or more active co-agents, or the controlled delivery from a drug delivery device or system of a therapeutically effective amount of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with one or more active co-agents.

21. A method for the prevention or reduction of vascular access dysfunction in association with the insertion or repair of an indwelling shunt, fistula or catheter, or actual treatment, in a subject in need thereof, which comprises administering a controlled delivery from a drug delivery medical device or system of a therapeutically effective amount of N-{5-[4-(4-methyl-piperazino-methyl)-benzoylamido]-2-methylphenyl}-4-(3-pyridyl)-2-pyrimidine-amine or a pharmaceutically acceptable salt or crystal form thereof, optionally in conjunction with one or more active co-agents.
